(19) United States
(12) Patent Application Publication
(10) Pub. No.: US 2026/0049971 A1
(43) Pub. Date: Feb. 19, 2026

(54) METHOD AND APPARATUS FOR MEASURING WATER POTENTIAL OF GRAVEL SOIL WITH AUTOMATIC WATER COLLECTION AND REPLENISHMENT SYSTEM

(71) Applicant: ZHEJIANG UNIVERSITY, Hangzhou (CN)

(72) Inventors: Qing LYU, Hangzhou (CN); Shiying YU, Hangzhou (CN); Junyu WU, Hangzhou (CN); Kailun SHEN, Hangzhou (CN); Zhenghua LIU, Hangzhou (CN)

(73) Assignee: ZHEJIANG UNIVERSITY, Hangzhou (CN)

(21) Appl. No.: 19/254,470

(22) Filed: Jun. 30, 2025

(30) Foreign Application Priority Data
Jun. 28, 2024 (CN) 202410859997.X

(51) Int. Cl. G01N 33/24

(52) U.S. Cl. CPC G01N 33/246

(57) ABSTRACT

Methods and apparatuses for measuring water potential in gravel soil with an automatic water collection and replenishment system are provided. The apparatus includes a tensiometer assembly configured to measure soil water potential in a gravel soil region, a replenishment water collection unit configured to collect and pre-treat water resources, and provide replenishment water to the tensiometer assembly, a replenishment water degassing unit configured to degas and deliver water filtered by the replenishment water collection unit to the tensiometer assembly, and a control unit configured to acquire a water level in the tensiometer assembly, control the replenishment water degassing unit to replenish water to the tensiometer assembly for tension measurement. The replenishment water collection unit continuously collects the replenishment water, and the control unit conveys the water from the replenishment water collection unit to the replenishment water degassing unit when a condition is fulfilled.

### CROSS-REFERENCE TO RELATED APPLICATIONS

This application claims priority to Chinese Patent Application No. 202410859997.X, filed on Jun. 28, 2024, the entire contents of which are incorporated herein by reference.

### TECHNICAL FIELD

The present disclosure relates to the technical field of testing or analyzing materials by measuring the chemical or physical properties of the materials, and in particular, relates to methods and apparatuses for measuring water potential in gravel soil with an automatic water collection and replenishment system.

### BACKGROUND

A soil tensiometer is a device used to measure soil water potential (or tension), which represents the energy state of water in the soil. Soil tensiometers are widely used in agriculture, environmental monitoring, and scientific research to help users understand soil moisture status and tension levels. However, in practical applications, tensiometers often fail due to water loss in the tube caused by dry monitoring environments or inadequate sealing. Measurement results can be inaccurate and unstable if non-degassed water is used. Additionally, traditional tensiometers overlook the influence of water column height in the tube on measurements, preventing accurate reflection of true soil water potential. Furthermore, when measuring water potential in gravel soil regions, vertical drilling for burial is impractical; instead, soil must be excavated to the measurement depth, and the tensiometer buried at an angle. This angled burial leads to uneven pressure on the tube body, potentially causing bending or rupture. Oblique installation also poses challenges, and during backfilling in complex geological conditions, improper handling may allow debris to damage the measuring tube.

Therefore, there is a need for methods and apparatuses for measuring water potential in gravel soil using an automatic water collection and replenishment system to enable efficient, convenient, and reliable monitoring of soil water potential dynamics.

### SUMMARY

One or more embodiments of the present disclosure provide an apparatus for measuring water potential in gravel soil with an automatic water collection and replenishment system. The apparatus includes a tensiometer assembly configured to measure soil water potential in a gravel soil region to be measured; a replenishment water collection unit configured to collect and pre-treat water resources, and provide replenishment water to the tensiometer assembly; a replenishment water degassing unit disposed between the tensiometer assembly and the replenishment water collection unit, and configured to degas and deliver filtered water to the tensiometer assembly; and a control unit operatively connected to the tensiometer assembly, the replenishment water collection unit, and the replenishment water degassing unit.

In some embodiments, the tensiometer assembly includes a soil contact head scaled into a lower opening of a measuring tube, with a combined sensor positioned in an upper portion of the measuring tube. The control unit obtains sensing data collected by the combined sensor, obtains a real-time value of the soil water potential, and replenishes water into the measuring tube when a preset condition is met via a water replenishment mechanism connected to the replenishment water degassing unit.

In some embodiments, the measuring tube includes a vertical tube and a horizontal tube. An arcuate tube is provided between the vertical tube and the horizontal tube. The vertical tube, arcuate tube, and horizontal tube are integrally formed, arranged sequentially, and spatially interconnected. The soil contact head is inserted into an end of the horizontal tube.

In some embodiments, the combined sensor includes a first liquid level sensor and a first pressure sensor disposed within the measuring tube. The water replenishment mechanism includes a first water pump disposed at a top of the measuring tube, and an outlet of the first water pump is connected to an inner cavity of the measuring tube.

In some embodiments, the apparatus further includes: a PLC chip configured to determine a predicted water potential value based on soil water potential values and soil properties in a preset time period. The preset condition includes the predicted water potential value being lower than a water potential threshold or a water potential change amplitude being lower than a water potential change threshold.

In some embodiments, the PLC chip is further configured to: determine the predicted water potential value based on the soil water potential values and the soil properties using a water potential prediction model, the water potential prediction model being a machine learning model.

In some embodiments, when the preset condition is that the predicted water potential value is lower than the water potential threshold or the water potential change amplitude is lower than the water potential change threshold, a volume of water replenished to the measuring tube by the water replenishment mechanism connected to the replenishment water degassing unit is determined based on a first difference between the predicted water potential value and the water potential threshold or a second difference between the water potential change amplitude and the water potential change threshold.

In some embodiments, the replenishment water collection unit includes a water collection tank. The water collection tank is equipped with a filter assembly and a second liquid level sensor, and an outlet of the water collection tank is connected to the replenishment water degassing unit via a second water pump. The control unit monitors a status of at least one of the second liquid level sensor and the replenishment water degassing unit, and controls opening and closing of the second water pump.

In some embodiments, the filter assembly includes a conical screen disposed at a top of the water collection tank, a filter screen is provided in the water collection tank under the conical screen, and an aperture of each filtering hole of the filter screen is smaller than an aperture of each filtering hole of the conical screen.

In some embodiments, the replenishment water degassing unit includes a degassing tank, and the degassing tank is equipped with a vacuum pump, a second pressure sensor, and an air valve. The control unit controls the vacuum pump to degas the degassing tank, monitors a degassing process through the second pressure sensor, and controls the air valve to open after completion of degassing to restore air pressure in the degassing tank.

In some embodiments, a rotary shaft is provided in the degassing tank, paddles are distributed outside the rotary shaft; and the rotary shaft is cooperatively set with the control unit.

In some embodiments, to control the vacuum pump to degas the degassing tank, monitor a degassing process through the second pressure sensor, and control the air valve to open after completion of degassing, the control unit is further configured to: in response to the second pressure sensor monitoring a decrease in a value of air pressure in the degassing tank to a preset value, control the vacuum pump to pause operation; control the second pressure sensor to collect barometric pressure data at a plurality of time points during the degassing process; and control the air valve to open at a preset rate based on the barometric pressure data.

In some embodiments, the replenishment water degassing unit further includes an ultrasonic vibration module. The control unit determines a previous sequence degassing volume based on a pressure difference before and after completion of a previous degassing operation, and determines, based on the previous sequence degassing volume, an ultrasonic parameter of the ultrasonic vibration module during a current degassing operation.

In some embodiments, the tensiometer assembly is set in the gravel soil region to be measured, a region outside the gravel soil region to be measured is excavated and backfilled with backfilled gravel soil, and the replenishment water collection unit and the replenishment water degassing unit is provided in a region where the backfilled gravel soil is located.

One or more embodiments of the present disclosure provide a method for controlling the apparatus for measuring water potential in gravel soil with automatic water collection and replenishment system, wherein the control unit acquires a water level in the tensiometer assembly in real time, and when the water level is lower than a preset level L1, controls the replenishment water degassing unit to replenish water to the tensiometer assembly until the water level reaches a preset level L2 to complete an automatic replenishment of water, 0<L1<L2; when the water level is between the preset level L1 and the preset level L2, a soil water potential value is measured; the replenishment water collection unit continuously collects replenishment water; and when the water in the replenishment water collection unit is higher than a preset level L3 or a liquid level in the replenishment water degassing unit is lower than a lowest liquid level, and the replenishment water degassing unit is in a non-degassing working state, the control unit conveys the water in the replenishment water collection unit to the replenishment water degassing unit, where L3>0.

### BRIEF DESCRIPTION OF DRAWINGS

The present disclosure will be further illustrated by exemplary embodiments, which will be described in detail by the drawings. These embodiments are not limiting, and in these embodiments, the same numeral denotes the same structure.

FIG. 1 is a schematic diagram of a structure of an apparatus for measuring water potential in gravel soil with an automatic water collection and replenishment system according to some embodiments of the present disclosure.

FIG. 2 is a schematic diagram of a structure of a measuring tube according to some embodiments of the present disclosure.

FIG. 3 is a schematic diagram of a structure of a degassing tank according to some embodiments of the present disclosure.

### DETAILED DESCRIPTION

In order to more clearly illustrate the technical solutions of the embodiments of the present disclosure, the drawings required to be used in the description of the embodiments will be briefly described below. Obviously, the drawings in the following description are only some examples or embodiments of the present disclosure, and it is possible for one skilled in the art to apply the present disclosure to other similar scenarios according to these drawings without creative labor. Unless obviously obtained from the context or the context illustrates otherwise, the same numeral in the drawings refers to the same structure or operation.

It should be understood that terms such as “system,” “device,” “unit,” and/or “module” as used herein are a way to distinguish between different components, elements, parts, sections, or assemblies at different levels. However, the words may be replaced by other expressions if other words accomplish the same purpose.

As shown in the present disclosure and the claims, unless the context clearly suggests an exception, the words “a,” “an,” and/or “the” do not refer specifically to the singular but may also include the plural. Generally, the terms “including” and “comprising” suggest only the inclusion of clearly identified steps and elements that do not constitute an exclusive list, and the method or apparatus may also include other steps or elements.

Before using a soil tensiometer, it is necessary to ensure that a hard tube of the soil tensiometer is filled with water and sealed. Then, the soil tensiometer is inserted into the soil, so that a clay head is in close contact with the soil, the water in the tube is in contact with the soil water through holes of the clay head, and gradually a balance (e.g., the water pressure in the tube is the same as that of the soil) is achieved. A pressure value in the tube monitored at this time is a water potential in the soil, which is also a matric suction force of the soil. The pressure value here is displayed through ways including, but not limited to, a mercury manometer, a vacuum pressure gauge, or directly transmitted from monitoring equipment to a data acquisition device. When the tensiometer is filled with water and buried in the soil, due to the porous clay head allowing only water to pass through under a certain air pressure, the water in the tensiometer slowly reaches a dynamic equilibrium with the soil water. The soil water enters and exits the tensiometer through capillary action, causing internal pressure changes. These changes are captured by sensor and converted into measurement values of soil water potential. The formula for determining the soil water potential satisfies:

$${ψ}_{m} = {ψ}_{s e n s o r} + ( {z}_{w a t e r} - {z}_{c u p} ) ,$$

where ψₘ denotes the soil water potential (usually negative), measured in meters (m); ψₛₑₙₛₒᵣ denotes a value of water potential measured by the sensor (usually negative), measured in meters (m); z_{water} denotes a height of a water level in the tensiometer, measured in meters (m); zₑᵤₚ denotes a height of the clay head of the tensiometer, measured in meters (m); and (z_{water}-z_{cup}) denotes the height of water column in the tensiometer, measured in meters (m). The negative value of the measured value indicates the soil water potential, and the absolute value of the soil water potential is a soil matric suction. As the soil dries up, the water potential decreases, and the soil matric suction increases. Conversely, when the soil moisture content increases (e.g., by irrigation or rainfall), the water potential increases and the soil matric suction decreases.

However, the tensiometers of the prior art suffer from three main disadvantages.

(1) In the field operation process, because the monitoring environment is often relatively dry and the insufficient airtightness of the tensiometer itself, the water in the tensiometer decreases due to evaporation and the air pressure inside the tube decreases to cause the external air to enter the tube, which affects the normal use of the tensiometer. A user must regularly check the air volume in the tube of the tensiometer. Once the air capacity exceeds ¾ or more of the tube volume of the tensiometer, it is necessary to fill up the tube with water again, so most of the tensiometers can only run continuously for 14-30 days. Especially when the monitoring point is in a remote location or when the monitoring region is relatively wide, the frequent refilling significantly increases the workload and cost, and it is not possible to accurately determine the time of refilling water, which is a major problem for the users. At the same time, degassed water is needed for the tensiometer, and in the case of using non-degassed water, with the decrease of the air pressure inside the measuring tube, the gas gradually separates out and forms a large number of bubbles, which can be attached to the tensiometer and lead to the inaccurate and unstable measurement results. However, the preparation of the degassed water is complex, and human intervention tends to increase the amount of dissolved gas during the refilling process. All these factors affect the accuracy of the measured results.

(2) The traditional tensiometer usually ignores the impact of the height of the water column in the tube on the measurement results, that is, the pressure sensor measurement value ψₛₑₙₛₒᵣ is simply equivalent to the soil water potential value ψₘ, which makes the tension measurement results cannot truly reflect the soil water potential. When the water column in the tube is too high, it may increase the water pressure inside the clay head, resulting in higher measurement results. Conversely, it may decrease the water pressure inside the clay head, resulting in lower measurement results. Especially when the water column inside the tube is high (the soil is moist and the water potential value is large), the deviation is more significant and frequent calibration is required.

(3) When it is necessary to measure the water potential in the gravel soil region and bury the tensiometer, it is not possible to bury the tensiometer by vertical drilling. Instead, it is necessary to excavate the soil to the depth of the measurement point and then bury the tensiometer obliquely. However, due to the insufficient compactness of the artificial backfill soil, a certain degree of settlement may occur after a period of rainfall leaching. The uneven settlement causes uneven compression of the tube body of the tensiometer, especially since the tube body is usually made of transparent plastic, which may cause the tensiometer to bend or break in severe cases. Moreover, there are installation difficulties with the inclined burial of the tensiometer. During the process of backfilling, due to the complex geological environment and improper operation, crushed stones may crush the measuring tube, resulting in damage to the tensiometer.

Therefore, some embodiments of the present disclosure provide methods and apparatuses for measuring water potential in gravel soil with automatic water collection and replenishment system to monitor and study the dynamics of the soil water potential in a highly efficient, convenient, and reliable manner, which is particularly suitable for the fields of agricultural irrigation management, ecological and geohazard research, soil protection, etc., thereby significantly enhancing the utility and efficiency of the soil tensiometers.

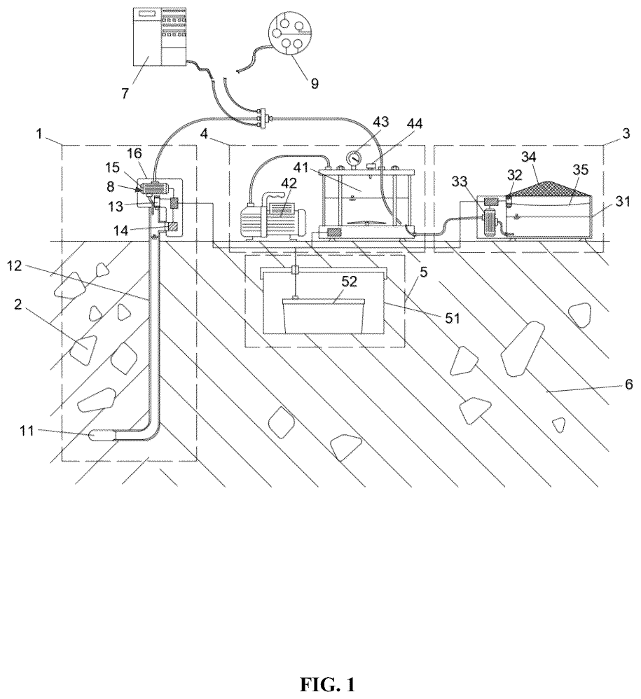

FIG. 1 is a schematic diagram of a structure of an apparatus for measuring water potential in gravel soil with an automatic water collection and replenishment system according to some embodiments of the present disclosure.

Some embodiments of the present disclosure provide an apparatus for measuring water potential in gravel soil with an automatic water collection and replenishment system (hereinafter referred to as the apparatus). As shown in FIG. 1, the apparatus includes: a tensiometer assembly 1 configured to measure soil water potential in a gravel soil region to be measured 2; a replenishment water collection unit 3 configured to collect and pre-treat water resources, and provide replenishment water to the tensiometer assembly 1; a replenishment water degassing unit 4, disposed between the tensiometer assembly 1 and the replenishment water collection unit 3, and configured to degas and deliver the filtered water to the tensiometer assembly 1, wherein the replenishment water degassing unit 4 cooperates with the tensiometer assembly 1 and the replenishment water collection unit 3; and a control unit 7 operatively connected to the tensiometer assembly 1, the replenishment water collection unit 3, and the replenishment water degassing unit 4.

The tensiometer assembly 1 may measure the soil water potential by measuring a suction force of the soil on water. The soil water potential refers to a physical parameter describing an energy state of the water in the soil, which reflects the attraction of soil moisture to plant roots. The soil water potential is also referred to as soil moisture tension. The magnitude of the soil moisture tension may be expressed in terms of negative pressure (in Pascal, Pa) or water head (in m). The smaller the value (the more negative) of the soil moisture tension, the greater the tension and the stronger the attraction of soil moisture to plants.

In practical applications, the measurement of the soil water potential is important for irrigation management, crop growth monitoring, soil moisture status assessment, etc. The measurement of the soil water potential can be used to guide agricultural production more scientifically, optimize the use of water resources, and improve crop yields and use efficiency of water.

The gravel soil region to be measured 2 refers to an operational region used to perform the soil water potential measurement.

The replenishment water collection unit 3 may be used to collect and pre-treat the water resource to ensure that the water quality of the water resource is suitable for use in the soil water potential measurement. The water resource may include rainwater, groundwater, or any other available water supply. Methods for pre-treating the water resource may include, but are not limited to, filtration, sedimentation, etc.

In some embodiments, the replenishment water collection unit 3 may also be used to provide replenishment water to the tensiometer assembly 1 to maintain accuracy and continuity of measurement.

The replenishment water degassing unit 4 may be used to degas the filtered water resource and replenish the degassed water resource to the tensiometer assembly 1. Methods for degassing the filtered water resource may include, but are not limited to, a vacuum degassing method, a thermal degassing method, a bursting method, etc.

The control unit 7 is the core of the apparatus and is responsible for coordinating and controlling the work of each of the above units. The control unit may include a microcontroller, a programmable logic controller (PLC), a personal computer (PC), a server, a mobile device (e.g., a smartphone), a cloud platform, or the like, or any combination thereof. In some embodiments, the control unit 7 may be communicatively coupled to other units of the apparatus to analyze and process data and/or information from the other units and control the other units.

It should be understood that the control unit 7 can be readily configured and implemented by those skilled in the art and may be customized as needed.

More descriptions regarding the apparatus may be found in related descriptions later.

Some embodiments of the present disclosure improve the accuracy of the soil water potential measurement by introducing an accurate water level monitoring and compensation mechanism for a measuring tube, which solves the measurement error due to the height of a water column. By equipping efficient water resource collection, degassing, and automatic water replenishment functions, the apparatus automatically generates and replenishes the degassed water, reducing the maintenance workload of the apparatus and ensuring a long-term stable operation of the apparatus.

In some embodiments, as shown in FIG. 1, the apparatus may further include a power supply module 5 configured to provide a working power for the tensiometer assembly 1, the replenishment water collection unit 3, and the replenishment water degassing unit 4. In some embodiments, the power supply module 5 is buried about 100 mm below the surface of the ground, reducing the above-ground footprint and facilitating battery replacement. The power supply module 5 consists of a waterproof protection box 51, a lithium battery 52, and wires. The waterproof protection box 51 serves to protect the lithium battery 52 from water and dust, and the lithium battery 52 provides a stable power for the tensiometer assembly 1, the replenishment water collection unit 3, and the replenishment water degassing unit 4.

In some embodiments, the apparatus may also include a solar panel. The solar panel is electrically connected to the power supply module 5. The solar panel is configured to provide power for the power supply module 5. Understandably, the solar panel can convert solar energy into electrical energy to continue to provide electrical power for the power supply module 5 to enhance the endurance of the apparatus.

In some embodiments, as shown in FIG. 1, the tensiometer assembly 1 includes a soil contact head 11 scaled into a lower opening of a measuring tube 12, with a combined sensor (a first liquid level sensor 13 and a first pressure sensor 14 as shown in FIG. 1) positioned in an upper portion of the measuring tube 12. The combined sensor may be set up in conjunction with the measuring tube 12 to facilitate the measurement. The control unit 7 obtains sensing data collected by the combined sensor, obtains a real-time soil water potential value (tension), and replenishes water into the measuring tube 12 when a preset condition is met via a water replenishment mechanism 8 coupled to the replenishment water degassing unit 4. The soil water potential value refers to a numerical representation of the soil water potential measured by the tensiometer assembly 1.

The soil contact head 11 refers to a component that transmits the suction force (water potential) of the soil moisture to the inside of the measuring tube 12 for measurement. In some embodiments, by the soil contact head 11 being in close contact with the soil, the soil water is brought into contact with water inside the measuring tube 12 through the bottom opening of the measuring tube 12 which is plugged and connected with the soil contact head 11, and gradually equilibrates, i.e., the water pressure inside the measuring tube 12 is the same as the water pressure of the soil. In the process, the sensing data collected by the combined sensor includes, but is not limited to, liquid level data within the measuring tube 12 for directing the water replenishment mechanism 8 to replenish water and pressure data for indicating the water pressure within the measuring tube 12. The control unit 7 obtains the sensing data of the combined sensor, further obtains the dynamic soil water potential value, and replenishes water into the measuring tube 12 via the water replenishment mechanism 8 when a preset condition is met.

The preset condition refers to a condition used to determine when to replenish water into the measuring tube 12. For example, the preset condition may include a soil water potential value being less than a water potential threshold. The water potential threshold may be set in advance by a technician based on experience, etc.

More descriptions regarding the preset condition and the water replenishment mechanism may be found in related descriptions later.

It is worth noting that all the joints of the units are bonded with a sealant to ensure the tightness and stability of the joints and to ensure that the interior of the tensiometer assembly 1 is completely scaled.

In some embodiments, the soil contact head 11 is configured to permit fluid communication between the interior and exterior of the measuring tube 12 while inhibiting excessive outflow from the measuring tube 12. The measuring tube 12 is formed of a material with high stiffness and corrosion resistance. Therefore, the soil contact head 11 may be set as a porous clay head, and the measuring tube 12 is made of stainless steel.

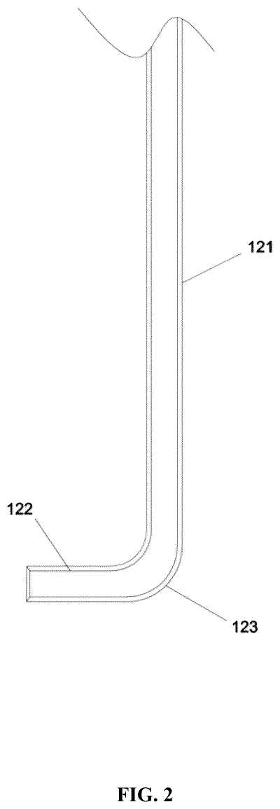

FIG. 2 is a schematic diagram of a structure of a measuring tube according to some embodiments of the present disclosure.

In some embodiments, as shown in FIG. 2, a measuring tube 12 includes a vertical tube 121 and a horizontal tube 122. An arcuate tube 123 is provided between the vertical tube 121 and the horizontal tube 122. The vertical tube 121, the arcuate tube 123, and the horizontal tube 122 are integrally formed and connected in sequence. The soil contact head 11 is inserted into an end of the horizontal tube 122.

In some embodiments, for a gravel soil region, by bending a bottom of the measuring tube 12 of the tensiometer assembly 1, a main body of the measuring tube 12 is kept in an upright state, effectively avoiding problems such as uneven pressure and installation difficulties, so as to realize long-term stable monitoring under complex geological conditions, and correct a soil water potential value by taking into account a (z_{water}-z_{cup}) value in the measured soil water potential value.

In some embodiments, as shown in FIG. 1, the combined sensor includes a first liquid level sensor 13 and a first pressure sensor 14 disposed within the measuring tube 12. The first liquid level sensor 13 and the first pressure sensor 14 are provided in conjunction with the measuring tube 12 to facilitate measurement. A water replenishment mechanism 8 includes a first water pump 15 disposed at a top of the measuring tube 12, and an outlet of the first water pump 15 is connected to an inner cavity of the measuring tube 12.

In some embodiments, the first liquid level sensor 13 (e.g., a laser liquid level sensor) is provided at the top of the measuring tube 12, which calculates and determines a height of a liquid level in real time by emitting a laser and irradiating the laser to a surface of the liquid, receiving a laser reflected by a receiver, and measuring a time delay of the laser. When a water level in the tensiometer assembly 1 is lower than a preset level L1, the first water pump 15 starts pumping water and injecting water into the measuring tube 12 until the water level reaches a preset level L2. In an implementation process, the first water pump 15 is connected to a degassing tank 41 through a rigid PVC pipe and is responsible for pumping the water in the degassing tank 41 into the measuring tube 12 or directly connecting the water in the degassing tank 41 to the municipal water supply and releasing it. More descriptions regarding the preset level may be found in related descriptions later.

In some embodiments, the first pressure sensor 14 is disposed outside of an upper tube sidewall of the measuring tube 12 and is capable of monitoring both negative water pressure and positive water pressure. For example, the first pressure sensor 14 may include a piczoresistive pressure sensor.

It is understood that a person skilled in the art may set up the combined sensor in an integrated manner in a plastic protective case 16, which serves to protect the combined sensor while facilitating setup.

It should be noted that in some embodiments of the present disclosure, for the detection of the liquid level, in addition to triggering the degassing of the replenishment water degassing unit 4 and replenishing degassed water into the measuring tube 12, it also serves to correct the measurement value.

In some embodiments, as shown in FIG. 1, the apparatus further includes a PLC chip 9 configured to determine a predicted water potential value based on soil water potential values and a soil properties in a preset time period.

The PLC chip 9 is configured to analyze and process data and/or information obtained from other units of the apparatus. In some embodiments, the PLC chip may be integrally provided in the control unit 7.

The soil water potential values in the preset time period refers to a set of all soil water potential values collected by the tensiometer assembly 1 during the preset time period. In some embodiments, the soil water potential values in the preset time period may be collected by the tensiometer assembly 1. The preset time period may be set in advance by the technician.

The soil property refers to a physical property, a chemical property, and a biological property of the soil. In some embodiments, the soil property may include a type of the soil. In some embodiments, the soil property may be obtained through input by the technician.

The predicted water potential value refers to a soil water potential value at a future time point or soil water potential values at a plurality of future time points in a future time period. The duration of the future time period and the duration of the preset time period may be the same or different. Time intervals of the plurality of future time points may be the same as monitoring intervals of historical water potential data.

The predicted water potential value may be determined in a plurality of ways. In some embodiments, the PLC chip 9 may determine the predicted water potential value by vector matching based on the soil water potential values and the soil properties in the preset time period. For example, a database may include a plurality of sets of reference vectors and actual soil water potential values corresponding to the plurality of sets of reference vectors. The reference vectors may be constructed based on historical soil water potential values and historical soil properties in the preset time period in historical data. The PLC chip may construct a to-be-matched vector based on the soil water potential values and the soil properties in the preset time period, perform a vector matching between the plurality of sets of reference vectors in the database and the to-be-matched vector, and identify an actual soil water potential value corresponding to a reference vector with the highest similarity with the to-be-matched vector as the predicted water potential value corresponding to the to-be-matched vector. Methods for determining the similarity include, but are not limited to, cosine similarity, Euclidean distance, etc.

According to some embodiments of the present disclosure, by setting the PLC chip 9 and determining the predicted water potential value based on the soil water potential values and the soil properties in the preset time period, it is possible to make timely judgments based on soil water potential values in the future time period, so as to take corresponding measures.

In some embodiments, the PLC chip 9 is further configured to determine the predicted water potential value based on the soil water potential values and the soil properties using a water potential prediction model.

The water potential prediction model refers to a model configured to determine the predicted water potential value. In some embodiments, the water potential prediction model is a machine learning model. For example, the water potential prediction model may include a deep neural network (DNN) model or any other customized model.

In some embodiments, an input of the water potential prediction model may include the soil water potential values and the soil properties in the preset time period, and an output of the water potential prediction model may include the predicted water potential value.

In some embodiments, the water potential prediction model may be obtained based on a plurality of training samples with labels. The training samples may include sample soil water potential values and sample soil properties in a first historical time period, and the labels may include actual soil water potential values at one or more time points in a second historical time period corresponding to the training samples. In some embodiments, the training samples may be determined based on historical data, and the labels may be obtained from actual measurements. The first historical time period is prior to the second historical time period.

In some embodiments, the PLC chip 9 may input the plurality of training samples with labels into an initial water potential prediction model. A loss function is constructed from the labels and the output of the initial water potential prediction model. Based on the loss function, parameters of the initial water potential prediction model are iteratively updated through gradient descent or other methods. The training is completed when a preset condition is met, and a trained water potential prediction model is obtained. The preset condition may be that the loss function converges, a count of iterations reaches a threshold, etc.

In some embodiments of the present disclosure, by the trained water potential prediction model, the predicted water potential value can be quickly and accurately determined so as to subsequently lay the groundwork for determining whether to replenish water for the tensiometer assembly 1.

In some embodiments, the preset condition further includes the predicted water potential value being below a water potential threshold or a water potential change amplitude being lower than a water potential change threshold.

The water potential change amplitude is used to reflect a change in the soil water potential value in the future time period. In some embodiments, the water potential change amplitude may be obtained by calculating a difference between a mean value of the predicted water potential values and a mean value of the soil water potential values in the preset time period. It should be noted that the water potential change threshold may be set in advance by the technician based on experience, etc.

In some embodiments, when the preset condition is that the predicted water potential value is lower than the water potential threshold or the water potential change amplitude is lower than the water potential change threshold, a volume of water replenished to the measuring tube 12 by the water replenishment mechanism 8 connected to the replenishment water degassing unit 4 is determined based on a first difference between the predicted water potential value and the water potential threshold or a second difference between the water potential change amplitude and the water potential change threshold.

The first difference refers to a difference between the predicted water potential value and the water potential threshold. The second difference refers to a difference between the water potential change amplitude and the water potential change threshold. In some embodiments, the water replenished volume may be positively correlated with the first difference or the second difference. For example, the larger the first difference, the larger the water replenished volume. As another example, the larger the second difference, the larger the water replenished volume.

In some embodiments of the present disclosure, by determining the predicted water potential value, a replenishment water process can be predicted in advance to determine whether the tensiometer assembly 1 needs to be replenished with water in advance, rather than waiting until the water level reaches the preset level L1 to replenish water violently at once, thereby reducing drastic fluctuation of the water level within the measuring tube 12, which in turn is favorable for improving continuity and accuracy of the measurement.

In some embodiments, as shown in FIG. 1, the replenishment water collection unit 3 includes a water collection tank 31, and the water collection tank 31 is equipped with a filter assembly and a second liquid level sensor 32. An outlet of the water collection tank 31 is connected to the replenishment water degassing unit 4 via a second water pump 33.

In some embodiments, the second liquid level sensor 32 (e.g., a laser liquid level sensor) on the water collection tank 31 may monitor the water level in the water collection tank 31 in real time. When the water level is higher than a preset water level, the second water pump 33 is activated to pump water in the water collection tank 31 to the degassing tank 41 of the replenishment water degassing unit 4, except when the replenishment water degassing unit 4 is in a degassing operating state. The control unit 7 can coordinate the overall operation of the apparatus. Exemplarily, when the replenishment water degassing unit 4 is in the degassing operating state, the second water pump 33 is controlled, through the control unit 7, to suspend operating until the replenishment water degassing unit 4 ceases to operate to ensure that degassed water in the degassing tank 41 is pure, thereby ensuring efficient operation of the apparatus.

In some embodiments, as shown in FIG. 1, the filter assembly includes a conical screen 34 disposed at a top of the water collection tank 31, and a filter screen 35 is provided in the water collection tank 31 under the conical screen 34. An aperture of each filtering hole of the filter screen 35 is smaller than an aperture of each filtering hole of the conical screen 34.

In some embodiments, the water collection tank 31 is configured to collect and pre-treat water resources. The water resources include, but are not limited to, the municipal water and the collected outdoor water resources (e.g., rainwater). The municipal water is generally unadulterated with visible impurities, whereas the collected outdoor water resource needs to be filtered. Access to the municipal water is readily understood by those skilled in the art, such as by setting up a connector in the sidewall of the water collection tank 31.

In some embodiments, the conical screen 34 may be made of stainless steel to prevent large debris (e.g., leaves) from falling into the water collection tank 31 and filter coarser particles of gravel. The filter screen 35 may filter finer particles of grit to ensure that the collected rainwater is purer.

It should be noted that the conical screen 34 may also be made of any other feasible material (e.g., plastic). The material of the filter screen 35 and the material of the conical screen 34 may be the same or different and are not limited herein.

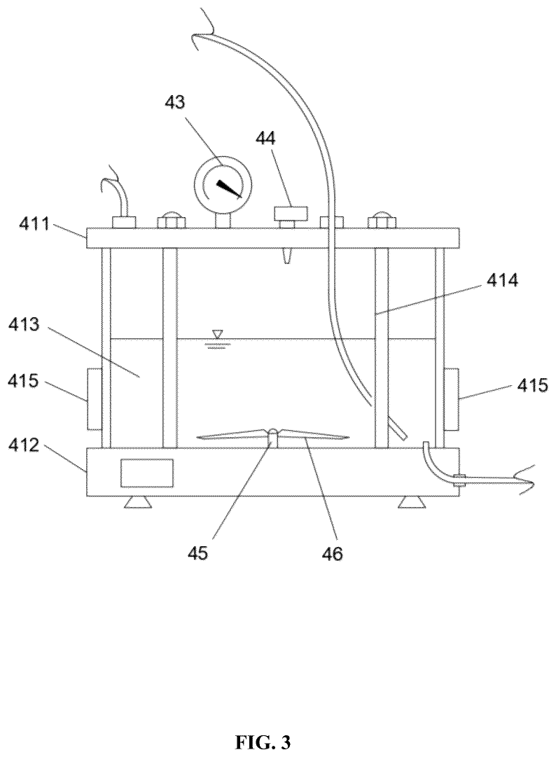

FIG. 3 is a schematic diagram of a structure of a degassing tank according to some embodiments of the present disclosure.

In some embodiments, as shown in FIG. 3, the replenishment water degassing unit 4 includes the degassing tank 41, and the degassing tank 41 is equipped with a vacuum pump 42, a second pressure sensor 43, and an air valve 44. The control unit 7 controls the vacuum pump 42 to degas the degassing tank 41, monitors a degassing process through the second pressure sensor 43, and controls the air valve 44 to open after completion of degassing to restore air pressure in the degassing tank 41. More descriptions regarding how the control unit controls the vacuum pump to degas the degassing tank, monitors the degassing process through the second pressure sensor, and controls the air valve to open after completion of the degassing may be found in related descriptions later.

In some embodiments, the principle of preparing the degassed water is to reduce the air pressure on the surface of rainwater, allowing gases dissolved in the liquid to escape. According to Henry's law, under low pressure conditions, since the solubility of gas is proportional to pressure, and a decrease in pressure leads to a decrease in solubility, the dissolved gases in the liquid under a vacuum environment begin to escape, forming bubbles and rising to the surface of the liquid. Based on the principle, the degassing tank 41 and the vacuum pump 42 that cooperates with the degassing tank 41 are provided.

In some embodiments, the degassing tank 41 comprises a stainless-steel top cover 411 and a bottom base 412, with a cylindrical glass sidewall 413 interposed therebetween. The assembly is hermetically sealed and structurally robust for operation under near-vacuum conditions (approaching 0 kPa absolute). The components of the degassing tank 41 are tightly connected as a whole with column bolts 414 and nuts. The second pressure sensor 43 is provided to obtain the air pressure inside the degassing tank 41. In some embodiments, the second pressure sensor 43 may be provided as a vacuum pressure gauge.

In some embodiments, the vacuum pump 42 may reduce and maintain the air pressure inside the degassing tank 41 at a range of 2 to 10 kPa (approximately 2%-10% of atmospheric pressure).

In some embodiments, as shown in FIG. 3, a rotary shaft 45 is provided in the degassing tank 41, paddles 46 are distributed outside of the rotary shaft 45, and the rotary shaft 45 is operatively connected to the control unit.

In some embodiments, in order to improve degassing efficiency, the rotary shaft 45 and the paddle 46 can cooperate to form a propeller blade. The control unit 7 controls the rotary shaft 45 to rotate to make the propeller blade agitate, and the agitation thereof can increase a surface area of the liquid (e.g., water) and a contact area of a gas-liquid interface, so as to accelerate the escape of the dissolved gases. Under a vacuum or near-vacuum environment, by continuously renewing the liquid surface, the dissolved gases can escape from the liquid more easily, thereby improving the degassing efficiency.

As shown in FIG. 3, the propeller blade may be provided at a bottom of the degassing tank 41 to agitate the liquid inside the degassing tank 41.

In some embodiments, to control the vacuum pump 42 to degas the degassing tank 41, monitor the degassing process through the second pressure sensor 43, and control the air valve 44 to open after completing of the degassing, the control unit 7 is further configured to: in response to the second pressure sensor 43 monitoring a decrease in a value of air pressure in the degassing tank 41 to a preset value, control the vacuum pump 42 to pause operation; control the second pressure sensor 43 to collect barometric pressure data at a plurality of time points during the degassing process; and control the air valve 44 to open at a preset rate based on the barometric pressure data.

Both the preset value and the preset rate may be set by the technician based on demand. In some embodiments, the preset value may be a preset air pressure P1.

The barometric pressure data refers to barometric pressure change data of the degassing tank 41 during the degassing process. For example, the barometric pressure data may include a value of air pressure and the air content.

In some embodiments, the control unit 7 may, based on the barometric pressure data, control the air valve 44 to open at the preset rate in response to the value of air pressure at a certain time point being lower than a preset value (e.g., the preset air pressure P1).

In some embodiments, the control unit 7 may determine, based on the barometric pressure data, a rate of reduction of air pressure; determine, based on the barometric pressure data and the rate of reduction of air pressure, a predicted time point corresponding to a time point when the value of air pressure is lower than the preset value; and in response to a current time point being the predicted time point, control the air valve 44 to open at the preset rate.

In some embodiments, the rate of reduction of air pressure may be obtained by calculation based on the values of air pressure in the barometric pressure data at a plurality of time points. For example, the rate of reduction of air pressure=(a value of air pressure at a later time point-a value of air pressure at a previous time point)/the time interval. It will be appreciated that any two of the plurality of time points have the same time interval.

In some embodiments, the control unit 7 may, based on the barometric pressure data and the rate of reduction of air pressure, determine the predicted time point corresponding to a time point when the value of air pressure is lower than the preset value by a plurality of means (e.g., analog simulation and linear fitting).

In some embodiments of the present disclosure, the control unit 7 collects the barometric pressure data at the plurality of time points during the degassing process via the second pressure sensor 43, and then controls the air valve 44 to open at a preset rate based on the barometric pressure data, which enables a more precise control, thereby favorably enhancing the degassing effect of the replenishment water degassing unit 4.

In some embodiments, as shown in FIG. 3, the replenishment water degassing unit 4 further includes an ultrasonic vibration module 415. The control unit 7 determines a previous sequence degassing volume based on a pressure difference before and after completion of the previous degassing operation, and determines an ultrasonic parameter of the ultrasonic vibration module 415 during the current degassing operation based on the previous sequence degassing volume.

The ultrasonic vibration module 415 is configured to promote the release of the dissolved gases in the liquid by vibration of ultrasonic waves, so as to improve the degassing efficiency. For example, the ultrasonic vibration module 415 may include a pneumatic ultrasonic generator, a piezoelectric ultrasonic generator, etc.

In some embodiments, the ultrasonic vibration module 415 may be provided at any feasible location outside the degassing tank 41. For example, the ultrasonic vibration module 415 may be provided at the sidewall or a bottom wall of the degassing tank 41. It is understood that the ultrasonic vibration module 415 may include one or more ultrasonic generators, and a specific count of the ultrasonic generators may be set based on actual demand.

The pressure difference before and after the end of the degassing operation refers to: a difference in air pressure in the degassing tank 41 at time points corresponding to before the start of the degassing operation and after the end of the degassing operation. In some embodiments, the control unit 7 may obtain the barometric pressure data in the degassing tank 41 at the time points corresponding to before the start of the degassing operation and after the end of the degassing operation by the second pressure sensor 43, and then obtain the pressure difference before and after the end of the degassing operation by making a difference.

The previous sequence degassing volume refers to a total amount of gas released in the previous degassing operation. In some embodiments, the control unit 7 may obtain the previous sequence degassing volume based on the pressure difference before and after the end of the previous degassing operation, combined with an amount of gas released from the water per unit of pressure difference reduction. The per unit of pressure difference may be 1 Pascal per second (1 Pa/s). In some embodiments, the amount of gas released from the water per unit of pressure difference reduction may be preset by the technician based on experience.

Merely by way of example, C=ΔP×L, where C denotes the previous sequence degassing volume, measured in cubic meters (m³); ΔP denotes the pressure difference before and after completion of the previous degassing operation, measured in pascals (Pa); and L denotes the amount of gas released from the water per unit of pressure difference reduction, measured in cubic meters per pascal (m³/Pa).

In some embodiments, the previous sequence degassing volume may also be obtained by actual measurement.

The ultrasonic parameter refers to a relevant parameter used to control the operation of the ultrasonic vibration module 415. In some embodiments, the ultrasonic parameter may include an ultrasound intensity, a vibration duration, etc. In some embodiments, the control unit 7 may determine the ultrasonic parameter of the ultrasonic vibration module during the current degassing operation by querying a preset table based on the previous sequence degassing volume. The preset table may include a plurality of sets of correspondences between the previous sequence degassing volumes and the ultrasonic parameters. In some embodiments, the preset table may be constructed based on historical data.

In some embodiments of the present disclosure, the replenishment water degassing unit 4 can effectively improve the degassing efficiency by the ultrasonic vibration module 415, which is favorable for improving the degassing effect of the replenishment water degassing unit 4. The control unit 7, by analyzing the total amount of gas released in the previous degassing operation and timely adjusting the ultrasonic parameter of the ultrasonic vibration module 415 during the current degassing operation, can enhance the degassing efficiency and at the same time save resources and cost.

In some embodiments, the tensiometer assembly 1 is set in the gravel soil region to be measured 2, a region outside the gravel soil region to be measured 2 is excavated and backfilled with backfilled gravel soil, and the replenishment water collection unit 3 and the replenishment water degassing unit 4 are provided in a region 6 where the backfilled gravel soil is located.

In some embodiments of the present disclosure, the replenishment water collection unit 3 and the replenishment water degassing unit 4 are provided in the region 6 where the backfilled gravel soil is located, which can minimize the influence of external factors on the measurement of the in-situ soil to ensure accuracy and reliability of the measured data.

Some embodiments of the present disclosure also provide a method for controlling an apparatus for measuring water potential in gravel soil with an automatic water collection and replenishment system, wherein the control unit acquires a water level in the tensiometer assembly 1 in real time. When the water level is lower than the preset level L1, the control unit controls the replenishment water degassing unit 4 to replenish water to the tensiometer assembly 1 until the water level reaches the preset level L2 to complete the automatic replenishment of water, 0<L1<L2. When the water level is between the preset level L1 and the preset level L2, a soil water potential value is measured. The replenishment water collection unit 3 continuously collects replenishment water. When the water in the replenishment water collection unit 3 is higher than a preset level L3 or a liquid level in the replenishment water degassing unit 4 is lower than a lowest liquid level, and the replenishment water degassing unit 4 is in a non-degassing working state, the control unit 7 conveys the water in the replenishment water collection unit 3 to the replenishment water degassing unit 4, where L3>0. The preset level L1, the preset level L2, and the preset level L3 may be set by the technician based on demand.

Exemplarily, a relatively complete embodiment is given below in conjunction with the control method described above. It will be appreciated that the embodiment is provided for reference only and is not limited.

The bottom of the tensiometer assembly 1 is a porous clay head with a diameter of about 30 mm, which serves as the soil contact head 11. The soil contact head 11 is connected to the measuring tube 12, which is made of high-hardness and corrosion-resistant stainless steel. The measuring tube 12 has a wall thickness of about 1 mm, a length of the vertical tube 121 is longer than 500 mm (an exact length depends on the soil depth of water potential to be measured), and the horizontal tube 122 is controlled to be about 100 mm. A piezoresistive pressure sensor capable of measuring negative pressure and positive pressure of water is disposed on the tube sidewall at a position of about 100 mm from the top of the measuring tube 12. The first water pump 15 and the laser liquid level sensor are mounted on the top of the measuring tube 12. The first water pump 15 is connected to the degassing tank 41 via a PVC rigid pipe with an inner diameter of 5 mm, and configured to pump water from the degassing tank 41 into the measuring tube 12 of the tensiometer assembly 1. The laser liquid level sensor monitors the height of the water column within the measuring tube 12 in real time.

When the liquid level in the tensiometer assembly 1 is lower than the preset level L1, a water replenishment command is issued. When the water replenishment command is received from the measuring tube 12, the air valve 44 is closed, the rotary shaft 45 rotates, the paddles 46 rotate, and the vacuum pump 42 begins to work. The value of air pressure in the degassing tank 41 is acquired in real time. When the value of air pressure drops to the preset air pressure P1, the air pressure in the degassing tank 41 is maintained at about the preset air pressure P1 for 30 minutes, and the water is degassed in a low air pressure environment. Then, the air valve 44 opens at the preset rate, and the air pressure inside the degassing tank 41 gradually returns to the atmospheric pressure, i.e., a round of degassing is completed. After the degassing is completed, the first water pump 15 starts pumping water from the degassing tank 41 to the measuring tube 12, replenishing the water until the liquid level within the measuring tube 12 reaches the preset level L2, at this time, the first water pump 15 stops pumping water, i.e., a round of automatic replenishment is completed. When the water level is in the range between the preset level L1 and the preset level L2, the measurement of the soil water potential value is carried out. The preset air pressure P1 may be set by the technician based on demand.

The replenishment water collection unit 3 and the replenishment water degassing unit 4 operate in concert. When the water level in the collection tank 31 exceeds the preset level L3, the second water pump 33 automatically delivers water to the degassing tank 41 to supply the degassing unit 4; however, if the degassing tank 41 is executing a degassing cycle at that time, the second water pump 33 remains off.

Beneficial effects that may be brought about by some embodiments of the present disclosure may include, but are not limited to, the following beneficial effects.

(1) The changes in the height of the water column in the tube are considered in the measurement result. By obtaining data on the changes in the height of the water column in the tube, real-time changes in the height of the water column in the tube are fully considered in the measurement results of the soil water potential, which significantly improves the accuracy and reliability of the measurement results.

(2) The ability of continuous monitoring is improved. The automatic water replenishment function makes the tensiometer assembly continuously operate for a longer period of time without frequent manual checking and replenishment of the water level, which is especially suitable for monitoring in remote regions or large areas. The stability of the monitoring data is improved. Interrupting measurements of the tensiometer due to the water level being too low is avoided by keeping the water level in a range of effective work, which ensures the continuity and stability of the monitoring data.

(3) The apparatus can automatically complete water resource collection and degassing treatment in the field, and replenish the degassed water into the tensiometer assembly, ensuring a continuous, low-disturbance supply of the degassed water in the tensiometer assembly. Through the automated water source management, the amount of maintenance workload is greatly reduced, and at the same time, the artificial disturbance to the degassed water is reduced, ensuring the long-term stable operation of the tensiometer.

(4) Human resources are saved, and the need for manual maintenance is reduced; therefore, the apparatus is especially suitable for long-term and large-scale soil tension monitoring projects. Compared to traditional soil tensiometers that need to replenish water to the tensiometer assembly every two weeks to a month for maintenance operations, the tensiometer assembly provided by the embodiments of the present disclosure only needs to be replenished once every five to six months, significantly extending the replenishment cycle and reducing the workload of the field.

(5) The apparatus has strong environmental adaptability, can work more stably under extreme climatic conditions (e.g., drought or rainy season), improve the adaptability and application range, and effectively avoid the problems of uneven pressure on the tensiometer assembly, installation difficulties, etc., so that the tensiometer assembly can operate stably in the gravel soil region and other regions not easy to drill holes.

(6) Wear and tear of the apparatus are reduced. By reducing the accumulation of sediment and other impurities in the contact head through frequent management of the water level, the life of the apparatus is extended, and the frequency of repairs and replacements is reduced.

The basic concepts have been described above, and it is apparent that to a person skilled in the art, the above detailed disclosure serves only as an example and does not constitute a limitation of the present disclosure. While not expressly stated herein, a person skilled in the art may make various modifications, improvements, and amendments to the present disclosure. Those types of modifications, improvements, and amendments are suggested in the present disclosure, so that those types of modifications, improvements, and amendments remain within the spirit and scope of the exemplary embodiments of the present disclosure.

What is claimed is:

1. An apparatus for measuring water potential in gravel soil with an automatic water collection and replenishment system, comprising:
a tensiometer assembly configured to measure soil water potential in a gravel soil region to be measured;
a replenishment water collection unit configured to collect and pre-treat water resources, and provide replenishment water to the tensiometer assembly;
a replenishment water degassing unit disposed between the tensiometer assembly and the replenishment water collection unit, and configured to degas and deliver filtered water to the tensiometer assembly; and
a control unit that operatively connected to the tensiometer assembly, the replenishment water collection unit, and the replenishment water degassing unit.

2. The apparatus according to claim 1, wherein the tensiometer assembly includes a soil contact head sealed into a lower opening of a measuring tube, with a combined sensor positioned in an upper portion of the measuring tube; and the control unit obtains sensing data collected by the combined sensor, obtains a real-time value of the soil water potential, and replenishes water into the measuring tube when a preset condition is met via a water replenishment mechanism connected to the replenishment water degassing unit.

3. The apparatus according to claim 2, wherein
the measuring tube includes a vertical tube and a horizontal tube,
an arcuate tube is provided between the vertical tube and the horizontal tube,
the vertical tube, the arcuate tube, and the horizontal tube are integrally formed, arranged sequentially, and spatially interconnected, and
the soil contact head is inserted into an end of the horizontal tube.

4. The apparatus according to claim 2, wherein the combined sensor includes a first liquid level sensor and a first pressure sensor disposed within the measuring tube; and
the water replenishment mechanism includes a first water pump disposed at a top of the measuring tube, and an outlet of the first water pump is connected to the interior of the measuring tube.

5. The apparatus according to claim 2, further comprising:
a PLC chip configured to determine a predicted water potential value based on soil water potential values and soil properties in a preset time period;
wherein the preset condition includes the predicted water potential value being lower than a water potential threshold or a water potential change amplitude being lower than a water potential change threshold.

6. The apparatus according to claim 5, wherein the PLC chip is further configured to:
determine the predicted water potential value based on the soil water potential values and the soil properties using a water potential prediction model, the water potential prediction model being a machine learning model.

7. The apparatus according to claim 5, wherein when the preset condition is that the predicted water potential value is lower than the water potential threshold or the water potential change amplitude is lower than the water potential change threshold, a volume of water replenished to the measuring tube by the water replenishment mechanism connected to the replenishment water degassing unit is determined based on a first difference between the predicted water potential value and the water potential threshold or a second difference between the water potential change amplitude and the water potential change threshold.

8. The apparatus according to claim 1, wherein the replenishment water collection unit includes a water collection tank, the water collection tank is equipped with a filter assembly and a second liquid level sensor, and an outlet of the water collection tank is connected to the replenishment water degassing unit via a second water pump; wherein
the control unit monitors a status of at least one of the second liquid level sensor and the replenishment water degassing unit, and controls opening and closing of the second water pump.

9. The apparatus according to claim 8, wherein the filter assembly includes a conical screen disposed at a top of the water collection tank, a filter screen is provided in the water collection tank under the conical screen, and an aperture of each filtering hole of the filter screen is smaller than an aperture of each filtering hole of the conical screen.

10. The apparatus according to claim 1, wherein the replenishment water degassing unit includes a degassing tank, and the degassing tank is equipped with a vacuum pump, a second pressure sensor, and an air valve, wherein
the control unit controls the vacuum pump to degas the degassing tank, monitors a degassing process through the second pressure sensor, and controls the air valve to open after completion of degassing to restore air pressure in the degassing tank.

11. The apparatus according to claim 10, wherein a rotary shaft is provided in the degassing tank, paddles are distributed outside the rotary shaft, and the rotary shaft is cooperatively set with the control unit.

12. The apparatus according to claim 10, wherein to control the vacuum pump to degas the degassing tank, monitor a degassing process through the second pressure sensor, and control the air valve to open after completion of degassing, the control unit is further configured to:
in response to the second pressure sensor monitoring a decrease in a value of air pressure in the degassing tank to a preset value, control the vacuum pump to pause operation;
control the second pressure sensor to collect barometric pressure data at a plurality of time points during the degassing process; and
control the air valve to open at a preset rate based on the barometric pressure data.

13. The apparatus according to claim 12, wherein the replenishment water degassing unit further includes an ultrasonic vibration module, wherein
the control unit determines a previous sequence degassing volume based on a pressure difference before and after completion of a previous degassing operation, and determines, based on the previous sequence degassing volume, an ultrasonic parameter of the ultrasonic vibration module during a current degassing operation.

14. The apparatus according to claim 1, wherein the tensiometer assembly is set in the gravel soil region to be measured, a region outside the gravel soil region to be measured is excavated and backfilled with backfilled gravel soil, and the replenishment water collection unit and the replenishment water degassing unit is provided in a region where the backfilled gravel soil is located.

15. A method for controlling the apparatus for measuring water potential in gravel soil with an automatic water collection and replenishment system according to claim 1, wherein
the control unit acquires a water level in the tensiometer assembly in real time, and when the water level is lower than a preset level L1, controls the replenishment water degassing unit to replenish water to the tensiometer assembly until the water level reaches a preset level L2 to complete an automatic replenishment of water, 0<L1<L2;
when the water level is between the preset level L1 and the preset level L2, a soil water potential value is measured;
the replenishment water collection unit continuously collects replenishment water; and
when the water in the replenishment water collection unit is higher than a preset level L3 or a liquid level in the replenishment water degassing unit is lower than a lowest liquid level, and the replenishment water degassing unit is in a non-degassing working state, the control unit conveys the water in the replenishment water collection unit to the replenishment water degassing unit, where L3>0.
